(19) United States
(12) Patent Application Publication
(10) Pub. No.: US 2026/0298904 A1
(43) Pub. Date: Oct. 1, 2026

(54) SYSTEMS AND METHODS FOR REAL-TIME TRANSLATION OF AQUATIC SENSOR MEASUREMENTS TO PROBABLE CAUSES VIA COMBINATION OF CAUSE-AND-EFFECT QUERIES AND LANGUAGE MODEL BASED SEMANTIC SIMILARITY

(71) Applicant: Prayrona Choudhury, Kennewick, WA (US)

(72) Inventor: Prayrona Choudhury, Kennewick, WA (US)

(21) Appl. No.: 19/489,891

(22) PCT Filed: May 24, 2024
(86) PCT No.: PCT/US2024/030978

§ 371 Date: Dec. 4, 2025

Related U.S. Application Data
(60) Provisional application No. 63/506,779, filed on Jun. 7, 2023.

(51) Int. Cl. G01N 33/18

(52) U.S. Cl. CPC G01N 33/18

(57) ABSTRACT

An aquatic environmental monitoring, analysis, and reporting system comprising a mobile waterborne craft equipped with a sensor subsystem for acquiring water-quality measurements. A first computing unit converts raw sensor signals into numerical measurement values, while a second computing unit executes program instructions implementing an environment analysis module that detects anomalies relative to expected parameter ranges. Upon anomaly detection, an inquiry module automatically generates natural-language queries and submits them to external search services, receiving textual results describing potential causes. An assessment module parses the results to extract cause-related terms, represents them in a numerical similarity space, and groups them into semantically similar cause clusters ranked as probable causes. A report generation module synthesizes the measurements, environmental conditions, and probable cause clusters into a human-readable diagnostic report transmitted in real time to a user device while the craft remains deployed, enabling end-to-end cause identification without laboratory sample processing.

An aquatic environmental monitoring, analysis, and reporting system comprising a mobile waterborne craft equipped with a sensor subsystem for acquiring water-quality measurements. A first computing unit converts raw sensor signals into numerical measurement values, while a second computing unit executes program instructions implementing an environment analysis module that detects anomalies relative to expected parameter ranges. Upon anomaly detection, an inquiry module automatically generates natural-language queries and submits them to external search services, receiving textual results describing potential causes. An assessment module parses the results to extract cause-related terms, represents them in a numerical similarity space, and groups them into semantically similar cause clusters ranked as probable causes. A report generation module synthesizes the measurements, environmental conditions, and probable cause clusters into a human-readable diagnostic report transmitted in real time to a user device while the craft remains deployed, enabling end-to-end cause identification without laboratory sample processing.

### FIELD OF THE INVENTION

The present invention relates generally to data collection and analysis in aquatic environments. More particularly, the invention relates to systems and devices for acquiring location-based data on water quality, contaminants, and flora presence and for processing such data using advanced algorithms to assess potential causes of water-quality issues and anomalies and to share those assessments across networked devices in substantially real time.

### BACKGROUND OF THE INVENTION

Aquatic biomes, including oceans, rivers, lakes, reservoirs, and ponds, play a critical role in maintaining the equilibrium of global ecosystems and supporting human activities such as agriculture, fisheries, and recreation. Many aquatic environments are undergoing rapid changes due to factors such as land-use changes, industrial development, and increased pollutant loads, sometimes beyond the natural adaptation capacities of resident organisms. These changes can degrade water quality, alter habitat suitability, and impair the reliability of water resources on which communities and industries depend.

To maintain healthy aquatic ecosystems and to support uses such as irrigation and fisheries, it is important not only to measure water-quality parameters but also to understand why anomalies occur. Traditionally, this understanding has relied on a combination of manual sampling, laboratory analysis, and expert interpretation of results, often with delays of days or weeks between data collection and diagnosis of potential causes. Such delays hinder timely responses to emerging problems.

Various systems have been proposed for automated or semi-automated monitoring of hydrology and water quality. For example, Chinese Patent Publ. No. CN103175513B describes a system for monitoring hydrology and water quality of a river basin utilizing the Internet of Things, including fixed and flowing sensors mounted in the river basin. Chinese Patent Publ. No. CN109470701A describes a system that performs biological monitoring of water treatment, including generating photos of pre-treated samples through an online monitoring device. These systems provide useful capabilities for measurement and, in some cases, visualization.

However, existing systems generally focus on detecting whether certain parameters exceed thresholds or fall outside predefined ranges. They do not provide automated, cause-centered analysis that mimics how an expert would investigate an anomaly, nor do they use cause-driven searching, vector-embedding methods, or other AI-based techniques to infer and rank potential causes of water-quality anomalies in real time at the point of data collection. As a result, users are often left with raw measurements and alarm flags but without a concise, actionable explanation of what conditions or activities are most likely responsible.

Accordingly, there remains a need in the art for a data collection and analysis system that can (i) acquire location-based aquatic environmental data using a mobile platform; (ii) process, store, and analyze that data using cause-driven searches and semantic grouping techniques; and (iii) provide system users with real-time assessments of potential causes of observed water-quality issues and anomalies, delivered while monitoring is still underway and without relying on laboratory processing of collected water samples.

### SUMMARY OF THE INVENTION

It is therefore an object of the present invention to assist environmental and scientific analysis of water-quality issues rapidly, on site, and using advanced algorithms to generate reliable and useful assessments for environmental analysts, regulators, and other stakeholders. A further object of the invention is to provide a system that, through repeated deployment and analysis, can improve its assessments over time by leveraging machine-learning and AI-based techniques.

The present invention provides an Aquatic Data Analysis and Reporting System, referred to herein as “AquaTranslate,” that combines a mobile aquatic rover with a software pipeline for translating numerical sensor measurements into human-readable explanations of likely causes. The rover is configured to traverse an aquatic environment and is equipped with water-quality sensors for measuring parameters such as temperature, pH, and total dissolved solids (TDS), which serve as indicators of water quality and potential contamination.

In preferred embodiments, AquaTranslate includes onboard electronics comprising one or more microcontrollers and a compact computing unit (for example, a Raspberry Pi-class device) that collect and forward numerical sensor measurements to an analysis engine. The analysis engine implements an environment analysis module that compares sensor values to expected ranges to identify anomalies, such as “high pH” or “elevated TDS.” When anomalies are detected, an inquiry module automatically generates one or more natural-language queries that encode the anomaly and relevant aquatic context and submits those queries to a web search engine or other external analysis service.

The system receives textual search results describing potential causes of the anomaly and processes the text using natural language processing techniques. An assessment module parses the results to extract cause-related terms, organizes the extracted terms into parameter-specific lists, and applies vector-based or semantic-similarity methods such as language-model-generated vector embeddings to group related terms into cause clusters. By comparing and clustering causes across different parameters, the system can identify convergent causal themes, for example industrial discharge, agricultural runoff, or natural geologic sources, that plausibly explain observed anomalies.

A report generation module then synthesizes the numerical measurements, their qualitative anomaly descriptors, and the most probable cause clusters into a concise, human-readable diagnostic report. In preferred embodiments, the report is transmitted from the rover or from a cloud-hosted engine to a user device via a wireless communication channel while the rover remains deployed, enabling on-site, real-time cause identification and reducing dependence on delayed laboratory analysis.

In this way, AquaTranslate streamlines both the collection and the interpretation of aquatic data. Rather than merely flagging that water is “out of range,” the system provides users with a structured explanation of what kinds of sources or conditions are most likely responsible, based on automated, cause-directed web searching and semantic grouping of the results. This capability can support more timely interventions, better-targeted field investigations, and improved management of aquatic resources.

Preferable embodiments of the AquaTranslate system include a rover and specialized software designed to analyze and process data collected from aquatic environments. The rover is responsible for gathering various types of aquatic data, including but not limited to temperature, pH, and TDS, along with associated location information. The software employs advanced algorithms to translate the collected data into structured reports and ranked lists of potential issues or concerns related to water quality. In representative implementations, the data-acquisition logic is implemented in C++ on Arduino microcontrollers for sensor measurements, while higher-level analysis, including natural language processing and querying of a web service or search engine, is implemented in Python.

In preferred embodiments, the software system for real-time analysis of anomalous water quality uses cause-driven searches to identify potential causes of anomalies. A breadth-first search strategy is used to generate multiple automated queries to a search engine, producing sets of search results that are then summarized and grouped using language-model-generated, vector-based comparisons to identify items with high semantic similarity. These vector embedding methods enable the system to group related causes and to highlight those causes that appear consistently across different parameters or query branches.

In some embodiments, the system further formulates follow-up queries to the web search engine based on newly discovered cause-related terms and groups potential causes using domain knowledge specific to aquatic or marine environments. Incorporating such domain knowledge helps refine cause clustering and improves the reliability and interpretability of the system's assessments and recommendations.

The hardware portion of the invention preferably includes a low-cost, stable aquatic vehicle (also referred to as a “rover”) using a pontoon-boat structure with onboard electrical and computing units. The electrical units may include brushed motors that drive “in-air” propellers, meaning propellers that act on air above the water surface rather than on the water itself. For a rover of this size, in-air propulsion can require less power and less expensive motors than underwater propellers, which face greater hydrodynamic drag and are more prone to entanglement.

In preferred embodiments, the rover is equipped with at least two motors so that a user on land can control speed and heading via a radio controller that independently adjusts each motor. The rover also carries one or more sensor units that include sensors for water temperature, pH, and total dissolved solids, in addition to a module for GPS data. Each sensor unit may be attached to a separate Arduino microcontroller that executes a C++ module for collecting sensor measurements.

A Raspberry Pi or similar compact computer is mounted onboard the rover to communicate with the Arduino microcontrollers and to execute software that translates sensor measurements into natural-language descriptions of potential causes. The Raspberry Pi system can be remotely controlled (for example, using a messaging framework), allowing a user on land to send commands to the on-board software to initiate or manage AquaTranslate analysis while the rover is in the water. Once probable causes have been generated from the sensor measurements, the AquaTranslate system notifies the user standing on land, such as on a riverbank, via a text message or other wireless communication. The system's analysis and messaging may include an assessment of the most likely causes of the observed conditions and, in some embodiments, suggested remedial actions or follow-up investigations.

The computing units used by the AquaTranslate system, such as Arduino microcontrollers and a Raspberry Pi, are selected and arranged to have minimal volume and weight, enabling attachment to an autonomous or human-guided rover without compromising stability. This compact design improves the rover's floating stability, reduces on-board power requirements, and extends the operating range and effective coverage area of the data collection and analysis system as a whole.

As those of skill in the art will appreciate, the present invention is not limited to the embodiments and arrangements described above. Other objects of the invention, as well as particular features, variations, and advantages, will be more apparent from consideration of the following brief description of the drawings and detailed description of the invention.

### BRIEF DESCRIPTION OF THE DRAWINGS

FIG. 1 is a schematic system diagram illustrating the Aqua Translate aquatic environmental monitoring system according to an embodiment of the present invention, showing a waterborne craft (100) with computing unit (10), sensor fusion module (12), cloud AI engine (14) comprising an aquatic environment analysis module, an inquiry module, and an assessment module, condition descriptors (16), messaging channel (18), wireless communication link (20), in-air propellers (22), dual-pontoon chassis (24), water-quality heatmap (26), and user device (30), along with a reference numeral table identifying system components.

FIG. 2 is a photograph of a hardware system according to an embodiment of the present invention, showing in-air propellers with brush motors (1), electronic speed controller and wireless receiver (2), GPS receiver module and navigation controller (3), first computing unit comprising microcontrollers (4), pH sensor (5), imaging subsystem comprising a camera (6), and dual-pontoon chassis made from PVC pipes (7).

FIG. 3 is a combined schematic and photographic illustration depicting real-world deployment of the AquaTranslate system, showing the waterborne craft (100) traversing an aquatic environment with computing unit (1), sensor fusion module (2), cloud-hosted AI engine (3), messaging channel (6), in-air propellers (7), dual-pontoon chassis (8), water-quality heatmap visualization (9), and user device (10), along with representative sensor data, diagnostic report output, and a reference numeral legend.

FIG. 4 is a photograph showing a top view of the data collection module circuit according to an embodiment of the present invention, with reference numerals identifying in-air propellers with brush motors (1), power source (2), electronic speed controller (3), and radio-controller signal receiver (4).

FIG. 5 is a schematic diagram illustrating the hardware connection architecture from sensors to the computing unit, showing a first microcontroller unit used to collect pH and TDS data, a second microcontroller unit (Raspberry Pi-class computing unit) used to receive sensor data, input the data into the AquaTranslate software, and transmit diagnostic results back to the user.

FIG. 6 is a flowchart illustrating an example step-by-step conversion of raw sensor data to computed results according to the method of the present invention, showing the sequential processing stages from data input through categorization, stem search generation, natural language processing extraction, list creation for TDS and pH measurements, and final output of common terms as suggested probable causes.

FIG. 7 is a schematic diagram illustrating iterative cause-driven query expansion performed by the inquiry module, showing (a) a pH query expansion tree branching from an initial query through extracted causal terms to explanatory text results, and (b) a dissolved oxygen query expansion tree demonstrating iterative branching to identify nitrogen and phosphorus as potential causal factors.

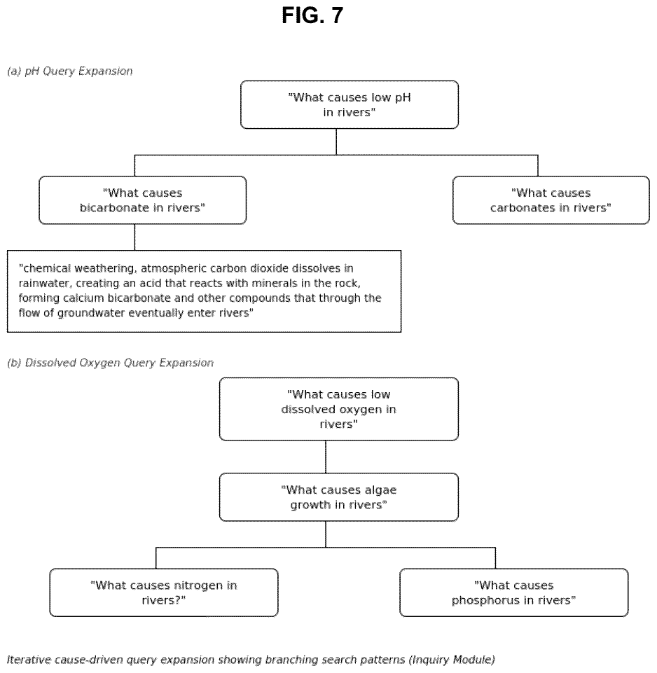

FIG. 8 is a schematic diagram illustrating iterative cause-driven query expansion for TDS (total dissolved solids) anomalies performed by the inquiry module, showing branching search paths from an initial query “What causes high TDS in rivers?” through intermediate causal terms including seawater intrusion and waste treatment chemicals, with dead-end detection and continued expansion through metal contaminants including zinc, lead, and copper.

FIG. 9 is a Venn diagram illustrating the assessment module's extraction of common factors using the principle of semantic similarity, showing overlapping candidate cause terms identified from pH-related searches and TDS-related searches, with the intersection representing related similar concepts including mining sites, iron sulfide, zinc, lead, copper, and cadmium that appear across multiple parameter anomalies.

FIG. 10 is a combined graph and aerial photograph illustrating guidance generated from a field experiment, showing (upper) a time-series plot of raw pH sensor counts during traversal of an aquatic environment with annotations indicating measurement locations including boat launch, dock areas, and areas of dense plant growth, and (lower) a satellite image of the test site showing the spatial relationship between measurement locations and observed water quality variations attributed to underwater stargrass (invasive species).

### DETAILED DESCRIPTION OF THE INVENTION

The following detailed description illustrates representative embodiments of systems and methods for real-time translation of aquatic sensor measurements into assessments of likely causes of detected conditions. The embodiments are described by way of example, not limitation. One of ordinary skill in the art will understand that various modifications and alternative configurations may be employed without departing from the scope of the invention as defined by the claims.

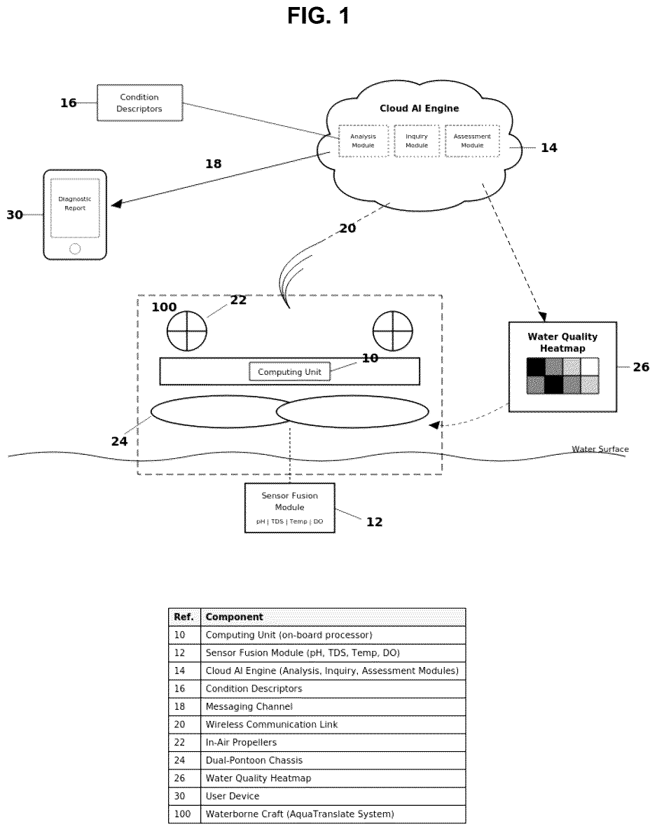

In general terms, preferred embodiments of the invention provide an aquatic data analysis system (“AquaTranslate”) that combines (i) a mobile waterborne craft configured to acquire geo-referenced water-quality measurements in situ and (ii) an automated, software-based analysis engine that converts numerical measurements into probable causes of water-quality anomalies in real time. As schematically illustrated in FIG. 1, a waterborne craft or rover floats at or near the water surface and carries on-board electronics including at least one computing unit and wireless communication hardware (collectively referenced at (1)). A sensor fusion module (2) is positioned such that one or more water-quality sensors are submerged in the aquatic environment during operation, enabling direct acquisition of chemical and physical measurements. The computing unit (1) communicates with a cloud-hosted AI engine (3) that implements an aquatic environment analysis module, an inquiry module, and an assessment module, as described in greater detail below. The cloud engine (3) may send alerts and explanatory messages to a user device via a messaging channel (6), and may also support generation of water-quality heatmaps (9) along the rover's path.

### Waterborne Craft and Sensor Configuration

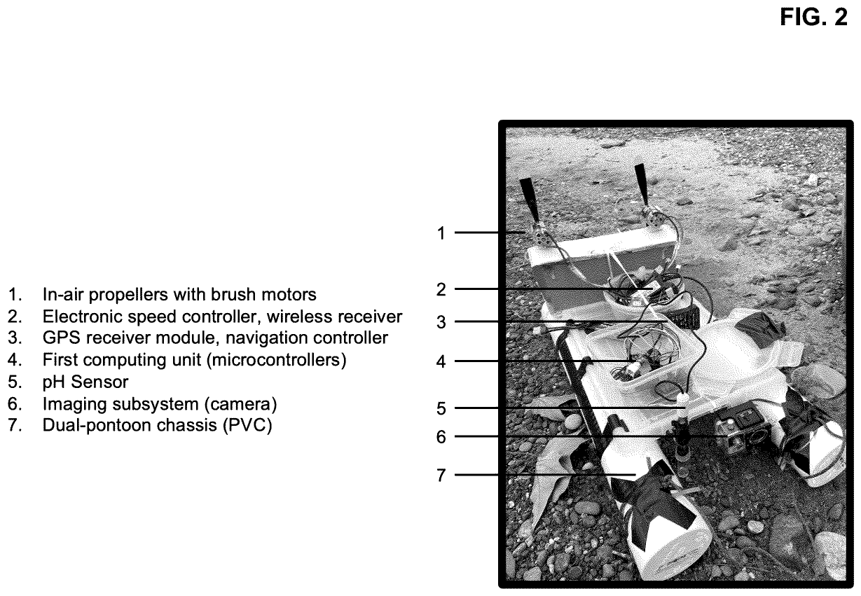

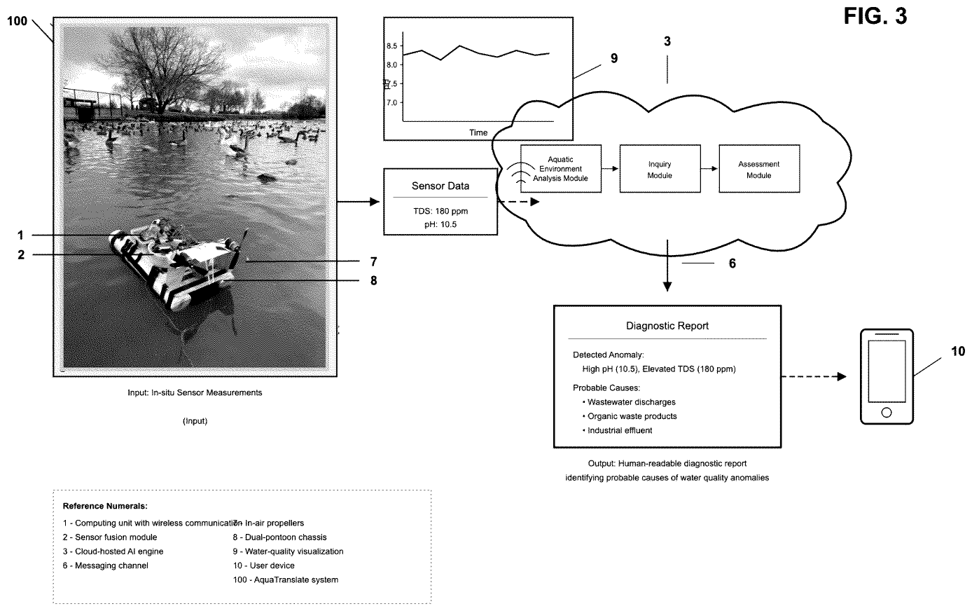

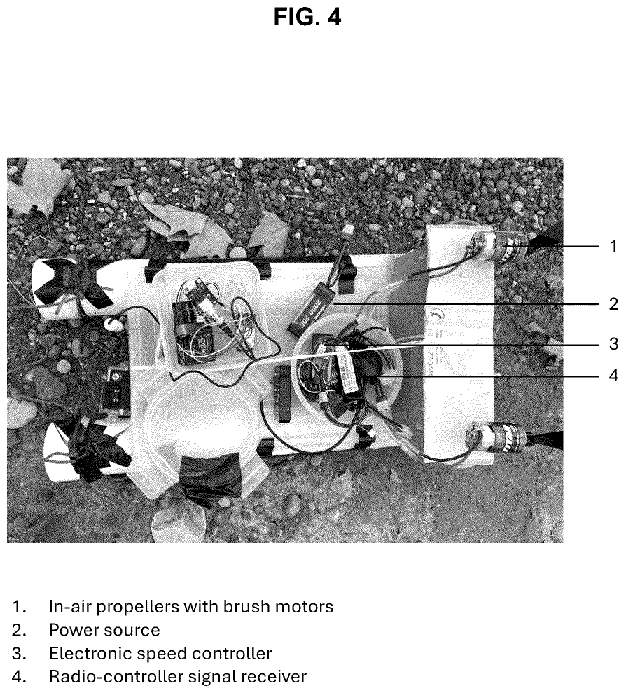

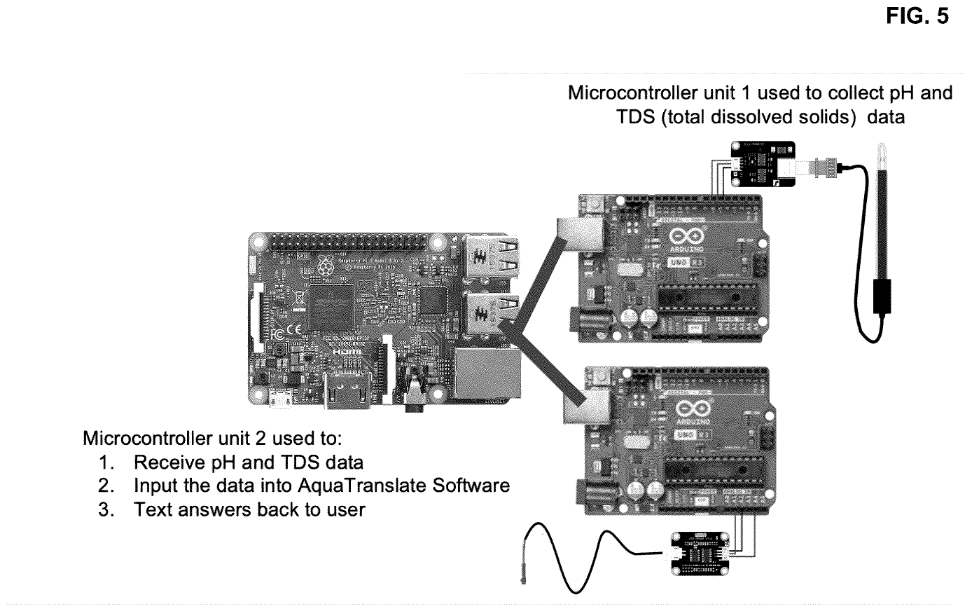

In one representative embodiment, shown in FIGS. 1-5, the waterborne craft comprises a dual-pontoon chassis (8), for example two generally cylindrical PVC tubes, mechanically joined by a central frame. The dual-pontoon configuration provides buoyancy and lateral stability in rivers, lakes, ponds, reservoirs, and other water bodies. The central frame supports a housing that encloses batteries, motor controllers, radio-frequency (RF) receivers, and computing elements. Brushed DC motors are mounted above the waterline and drive in-air propellers (7). Positioning the propellers in air, rather than underwater, reduces hydrodynamic drag on the propulsion system and enables use of relatively low-cost motors and batteries while still providing sufficient thrust to move the craft through the aquatic environment.

In some embodiments, two independent propulsion motors are provided, each driving a corresponding propeller on one side of the chassis. By varying the relative speeds of the motors, the craft can be steered through differential thrust. A user may control the rover directly from shore using a handheld radio controller coupled to a receiver mounted on the central frame. In alternative embodiments, an on-board navigation controller, such as a Pixhawk-class flight controller, receives a set of waypoints and autonomously guides the rover along a survey path.

The sensor fusion module (2) is mechanically mounted such that one or more water-quality sensors are fully immersed below the surface during operation. In various embodiments, the sensor fusion module includes pH sensors, temperature sensors, conductivity and/or total dissolved solids (TDS) sensors, dissolved oxygen sensors, turbidity sensors, or other chemical and physical sensors suitable for aquatic monitoring. A GPS receiver may be mounted on the rover to provide position information, which can be associated with each measurement to generate geo-referenced data. In some embodiments, the sensor fusion module is implemented as a removable probe assembly that can be swapped or reconfigured for different monitoring campaigns.

Sensor electronics may be implemented using one or more microcontrollers. In one embodiment, a first Arduino-class microcontroller is dedicated to pH acquisition and a second Arduino-class microcontroller is dedicated to TDS or conductivity acquisition. Each microcontroller executes a data-collection routine that periodically samples the attached sensor, applies calibration coefficients, associates the reading with a timestamp and, if available, GPS coordinates, and transmits the resulting measurement record to a higher-level computing unit. Transmission may occur via wired serial, USB, I²C, SPI, or other digital communication interfaces.

A Raspberry Pi-class computing unit, also mounted on the rover, receives measurement records from the microcontrollers and serves as an edge gateway for the Aqua Translate system. In a representative configuration, the Raspberry Pi logs continuous measurements to local storage for archival purposes and runs software that packages selected measurements for transmission to the cloud-based AI engine (3) via Wi-Fi, cellular, or other wireless networking. The Raspberry Pi may additionally provide local visualization through an on-board display or camera stream, and may send status information (for example, battery level, signal strength, or sensor health) to the user device.

Although a specific rover geometry and sensor layout are described for clarity, the invention is not limited to any particular craft or sensor platform. In alternative embodiments, the sensors may be mounted on a single-hull boat, a tethered buoy, a fixed station, an autonomous underwater vehicle, or a human-portable probe, provided that measurements can be transmitted to the aquatic environment analysis, inquiry, and assessment modules as described herein.

### Aquatic Environment Analysis Module

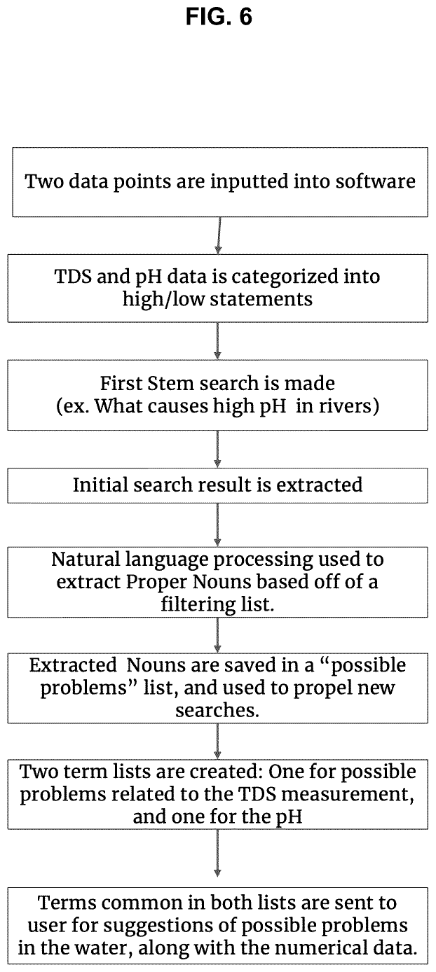

Preferable embodiments of the aquatic environment analysis module receive streams of numerical sensor measurements and transform them into qualitative descriptors that characterize the state of the aquatic environment. Referring to FIGS. 1, 6, and 8, the aquatic environment analysis module may execute on the on-board computing unit (1), the cloud AI engine (3), or a combination thereof. Each incoming measurement is associated with metadata including parameter type (for example, pH, TDS, temperature, dissolved oxygen), measurement value, timestamp, and location.

For each parameter, the analysis module consults stored parameter-specific reference ranges or thresholds that may be selected based on the type of water body (for example, river, lake, pond, estuary) and, in some embodiments, known regulatory standards or user-defined limits. Using these reference values, the module maps each numerical measurement into a qualitative category such as “normal,” “low,” “very low,” “high,” or “very high.” For example, a pH measurement of 10.5 in a river may be categorized as “high pH,” while a TDS measurement exceeding a stored threshold may be categorized as “elevated TDS.”

The aquatic environment analysis module may further detect anomalies by identifying measurements that cross thresholds, deviate from historical baselines, or exhibit abrupt temporal or spatial changes. In some embodiments, the module computes rolling averages, gradients, or other statistical indicators and flags measurement sequences that satisfy anomaly criteria. For each detected anomaly, the module generates an anomaly record that includes the parameter identifier, the qualitative descriptor (for example, “high pH”), and contextual information such as water-body type and location.

Anomaly records are provided as inputs to the inquiry module. In some embodiments, the analysis module filters anomalies to limit the number of concurrent inquiries for example by prioritizing anomalies with the largest deviation from normal ranges or those occurring near sensitive sites. In other embodiments, all anomalies are forwarded in parallel subject to resource constraints of the inquiry module.

### Inquiry Module and Cause-Driven Query Generation

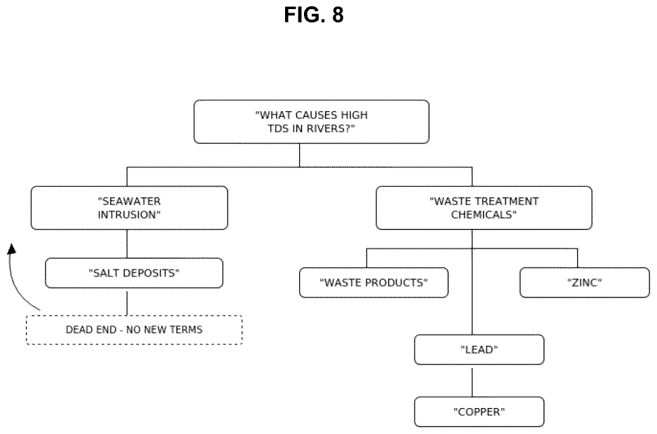

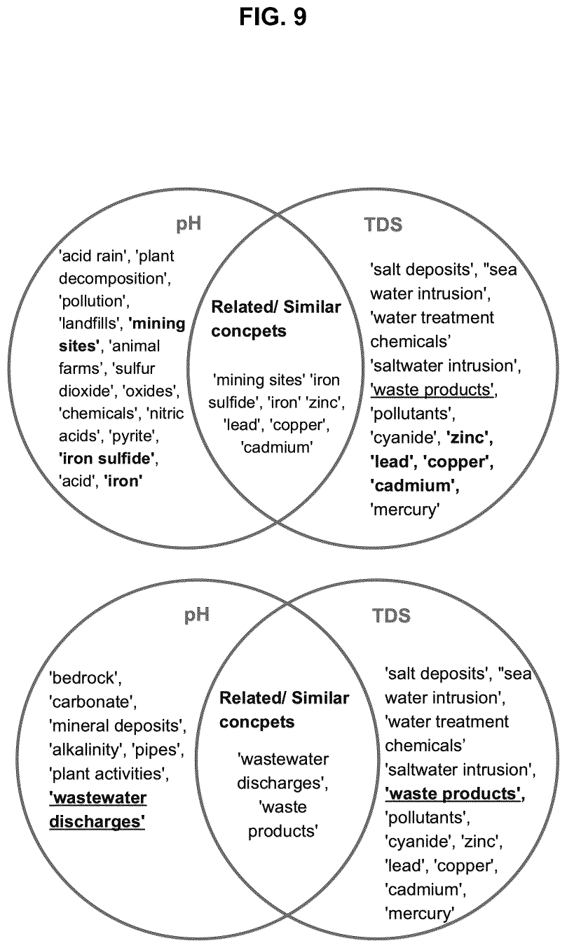

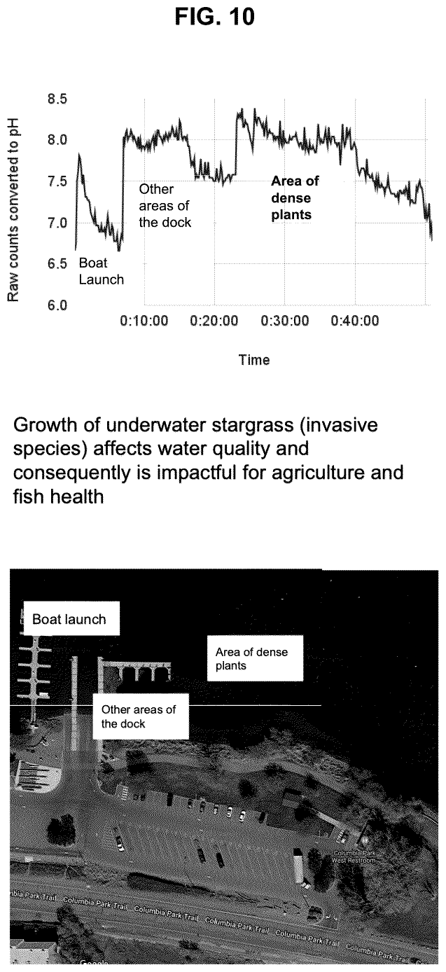

The inquiry module is configured to transform anomaly records into cause-driven search queries and to iteratively explore potential causes in a manner analogous to how a human expert would investigate an unexplained condition. As schematically illustrated in FIGS. 8-11, the inquiry module preferably employs a standardized query template that is domain-specific to aquatic environments to generate initial search strings. In one embodiment, the template is of the form:
“What causes”+[qualitative descriptor]+“in”+[aquatic context],
where the qualitative descriptor is taken from the anomaly record (for example, “high pH,” “elevated TDS”) and the aquatic context describes the environment (for example, “rivers,” “lakes,” “reservoirs,” or more specific phrases such as “urban rivers” or “irrigation canals”). This domain-specific formulation ensures that returned search results are relevant to aquatic conditions rather than generic or unrelated contexts.

Using this template, an anomaly record indicating high pH in a river yields an initial query string such as “What causes high pH in rivers?”. Additional contextual terms such as “near agricultural fields” or “near a marina” may be appended where available. The inquiry module submits these initial query strings to one or more search engines through an application programming interface (API) and receives result pages or snippets containing explanatory text.

The inquiry module then performs an iterative branching process to expand and refine the search space. In one preferred embodiment, the module applies a natural language processing (NLP) library to the returned text to identify nouns, noun phrases, or other expressions that plausibly represent causal factors related to the anomaly. Illustrative extracted terms for a high-pH anomaly might include “industrial discharge,” “mineral deposits,” “alkaline soil,” “wastewater effluent,” or “cleaning chemicals.”

Each extracted term is added to a parameter-specific candidate cause list associated with the originating anomaly. The inquiry module also uses selected extracted terms as branching variables to generate additional search queries by substituting them into the template. For example, the terms “wastewater effluent” and “cleaning chemicals” may produce secondary queries such as “What causes wastewater effluent in rivers?” and “What causes cleaning chemicals in rivers?”. These secondary queries are submitted to the search API, generating additional result snippets that are again processed by the NLP routines to extract further candidate cause terms.

In preferable embodiments, the branching process proceeds in a breadth-first manner for a bounded number of iterations. The inquiry module may, for example, limit the number of branching levels to between three and five, or apply other stopping criteria such as detecting that successive snippets are highly similar to previously processed text, that no new candidate cause terms are being discovered, or that a maximum query budget has been reached. Throughout this process, the module maintains, for each parameter or anomaly, a record of all queries issued, snippets received, and candidate cause terms extracted.

To manage redundancy and improve efficiency, the inquiry module may merge similar candidate cause terms within a given list using basic normalization (for example, stemming, lemmatization, synonym replacement) before passing them to the assessment module. In some embodiments, the module also associates each candidate term with simple scores, such as frequency of occurrence across snippets or recency of discovery, which can later be used to weight terms in the assessment stage.

### Assessment Module and Semantic Similarity Grouping

The assessment module receives the parameter-specific candidate cause lists from the inquiry module and is configured to summarize them, group them by semantic similarity, and identify convergent causal themes that plausibly explain one or more anomalies. The assessment module may first perform exact or near-exact string matching to identify candidate causes that appear in multiple lists (for example, in both a pH list and a TDS list). Such overlapping terms often correspond to sources that influence multiple parameters, such as “mine drainage,” “metal contamination,” or “sewage discharge.”

In preferred embodiments, the assessment module further employs vector-based similarity methods or language models to group candidate causes into semantic clusters. In one approach, the module transforms each candidate cause term, or longer excerpt of explanatory text, into a numerical vector representation using an embedding model. Standard clustering or nearest-neighbor algorithms may then be applied to group vectors that lie close to one another in a high-dimensional similarity space. Alternatively, the assessment module may query a large language model, such as OpenAI's davinci model or other suitable language models, with the candidate cause lists and instructions to cluster related terms into a small number of concept groups.

The resulting clusters represent candidate causal groups such as “industrial discharge,” “agricultural runoff,” “natural geologic sources,” or “organic waste from wildlife.” For each cluster, the assessment module may compute a confidence score based on factors such as (i) the number of distinct parameters for which terms in the cluster appear, (ii) the frequency with which constituent terms are observed across different search snippets, and (iii) the degree of similarity among terms in the cluster.

The assessment module then generates an assessment for one or more anomalies by ranking the causal groups according to their confidence scores and relevance. In illustrative embodiments, the module composes a short natural-language explanation that includes (i) the qualitative description of the anomaly (for example, “high pH and elevated TDS”), (ii) the top one or more causal groups (for example, “industrial or mining-related discharges high in dissolved minerals”), and (iii) a brief summary of the reasoning, such as noting that several candidate causes related to metals, mine drainage, and industrial effluent appeared across both pH and TDS search results.

The assessment module may additionally incorporate simple rules or models to align candidate cause groups with known field conditions. For example, if the rover's location is known to be near agricultural land, the system may increase the weight given to clusters related to fertilizer runoff or irrigation return flows. In other embodiments, the system may incorporate local regulatory or historical information to further refine candidate cause rankings.

### Reporting, Mapping, and Real-Time Operation

Preferably, the Aqua Translate system generates reports and visualizations while the waterborne craft is still traversing the aquatic environment. Referring again to FIGS. 1, 6, 9, and 10, assessment outputs may be transmitted from the cloud AI engine (3) to a user device via the messaging channel (6) as text messages, emails, dashboard updates, or alerts within a dedicated application. Each message may include at least a portion of the explanatory assessment, the associated location, and the time of detection.

In some embodiments, a mapping module aggregates geo-referenced measurements and their associated qualitative descriptors and assessments to form thematic maps or heatmaps of the monitored water body. Individual measurement points may be interpolated across a grid or represented as color-coded symbols indicating categories such as “normal,” “elevated,” or “critical” for one or more parameters. The mapping module may similarly display locations where specific causal groups (for example, “wastewater-related sources” or “metal contamination”) are assessed as likely, allowing rapid visual identification of hotspots and patterns along the rover's path.

The system may also support interactive queries initiated by the user. For example, a user may request further detail for a particular location or anomaly, prompting the assessment module to provide extended explanations or to re-run portions of the inquiry process with additional user-specified context. In some embodiments, the inquiry and assessment modules may be configured to run both in batch mode (processing stored data) and streaming mode (processing newly arriving data in real time).

### Implementation Considerations and Alternatives

The aquatic environment analysis, inquiry, and assessment modules described above may be implemented in a variety of programming languages and software architectures. In one embodiment, data acquisition and basic categorization logic execute in C++ or Python on the on-board computing unit, while web searching, snippet parsing, natural language processing, and semantic clustering execute on a cloud server. In other embodiments, the entire pipeline executes on an edge device mounted on the rover, or within a local base station, where sufficient computing resources are available.

NLP and clustering functionality may be realized using any suitable software capable of extracting terms and grouping them by meaning. For example, the system may employ open-source NLP libraries to identify parts of speech and extract candidate terms, commercial or open-source vector-embedding models to produce numerical representations of text, and standard clustering algorithms such as k-means, hierarchical clustering, or density-based clustering. Large language models can be accessed through APIs provided by third-party services. The specific choice of tools and models is not limiting; the inventive concepts reside in the structured generation of cause-driven queries, the iterative branching based on extracted terms, and the semantic convergence of candidate causes across parameters.

The particular sensors, water bodies, and anomaly types described herein are illustrative. The AquaTranslate system may employ any combination of chemical, physical, or biological sensors suitable for characterizing an aquatic environment, including but not limited to sensors for nitrate, phosphate, turbidity, chlorophyll, dissolved metals, or biological proxies. The same inquiry and assessment principles may also be applied to other environmental or industrial domains where numerical sensor measurements must be translated into plausible causes, such as air-quality monitoring, soil-health assessment, or process control in industrial plants.

While preferred embodiments have been described in conjunction with the illustrated figures, it will be appreciated that numerous variations and alternative configurations may be adopted without departing from the scope of the present invention. For example, the rover platform may be replaced with fixed or drifting sensor nodes, multiple rovers may cooperate to map large areas simultaneously, or the inquiry and assessment modules may operate on historical datasets rather than real-time streams. Accordingly, the embodiments described above are to be considered in all respects as illustrative and not restrictive, and the scope of the invention is defined by the appended claims rather than by the foregoing description.

What is claimed is:

1. An aquatic data analysis system comprising:
a waterborne craft navigable through an aquatic environment, the waterborne craft comprising one or more sensors for acquiring data on the aquatic environment;
an aquatic environment analysis module comprising one or more algorithms for analyzing anomalies in the aquatic environment by processing the data on the aquatic environment acquired by the one or more sensors of the waterborne craft, assessing the associated water and other aquatic material, and identifying anomalies found therein;
an inquiry module comprising one or more algorithms for generating one or more queries to identify potential causes of the anomalies identified by the aquatic environment analysis module, running the one or more queries on one or more internet search engines, and accumulating the results of the one or more queries; and
an assessment module comprising one or more algorithms for summarizing the results of the one or more queries using language model generated vector-based comparisons, grouping the potential causes of the anomalies utilizing accumulated system knowledge specific to aquatic environments into one or more causal groups, and generating an assessment of the one or more causal groups;
wherein the one or more sensors of the waterborne craft are in electronic communication with at least one of the aquatic environment analysis module, the inquiry module, and the assessment module enabling real-time operation of the aquatic data analysis system as the waterborne craft navigates throughout the aquatic environment.
